(19) United States
(12) Patent Application Publication
(10) Pub. No.: US 2023/0172594 A1
(43) Pub. Date: Jun. 8, 2023

(54) PUNCTURING DEVICE COMPRISING A DISTAL BALLOON FEATURE

(71) Applicant: Boston Scientific Medical Device Limited, Galway (IE)

(72) Inventors: Matthew DiCicco, Mississauga (CA); Gareth Davies, Mississauga (CA); John Paul Urbanski, Mississauga (CA); Eduardo Moriyama, Mississauga (CA); Patrick Ryan, Mississauga (CA); Daniel Wing Fai Mok, Mississauga (CA)

(21) Appl. No.: 18/154,183

(22) Filed: Jan. 13, 2023

Related U.S. Application Data
(63) Continuation of application No. PCT/IB2021/056256, filed on Jul. 12, 2021.
(60) Provisional application No. 63/051,106, filed on Jul. 13, 2020.

(51) Int. Cl. A61B 17/00; A61B 90/00

(52) U.S. Cl. CPC A61B 17/00234; A61B 90/39; A61B 2017/00247; A61B 2090/3966

(57) ABSTRACT

A method and apparatus are disclosed for a device for creating a puncture in a tissue. The puncturing device comprising an elongate member having a puncturing portion at the distal tip. The puncturing device further includes at least one inflatable feature positioned around the outer circumference and at a distal portion of the elongate member. The puncturing device includes a lumen extending from a proximal end of the elongate member to the inflatable feature. The inflatable feature is in a fluid communication with the lumen and can be inflated to dilate the puncture in the tissue or anchor the puncturing device in a heart structure.

### CROSS REFERENCE TO RELATED APPLICATION

This application is a continuation application of International Application No. PCT/IB2021/056256, filed Jul. 12, 2021, titled “A PUNCTURING DEVICE COMPRISING A DISTAL BALLOON FEATURE,” which claims priority to U.S. Provisional Application No. 63/051,106, filed Jul. 13, 2020, titled “A PUNCTURING DEVICE COMPRISING A DISTAL BALLOON FEATURE,” the entire disclosures of which are incorporated herein by reference.

### TECHNICAL FIELD

The disclosure relates to a transseptal puncturing device with means to enable an easier and safer sheath crossing of the septum. More specifically, the invention relates to a device and method for creating a puncture in the atrial septum using a transseptal puncturing device with a distal inflatable feature. The distal inflatable feature may be used for dilating the septum prior to sheath crossing or for anchoring the puncturing device in the pulmonary vein to act as a guiderail, which in turn prevents unpredictable sheath jumping.

### BACKGROUND OF THE ART

Certain medical procedures require the use of a medical device that can create punctures or channels through tissues of the heart. Specifically, puncturing the septum of a heart creates a direct route to the left atrium where numerous cardiology procedures take place. One such device that gains access to the left atrium is a transseptal puncturing device which, in some devices, delivers radiofrequency energy from a generator to the tissue to create the puncture. The user positions the puncturing device at a target location on the fossa ovalis located on the septum of the heart and turns on the generator to begin delivering energy to the target location. The delivery of radiofrequency energy to a tissue results in vaporization of the intracellular fluid of the cells which are in contact with the energy delivery device. Ultimately, this results in a void, hole, or channel at the target tissue site.

When performing a transseptal procedure to gain access to the left atrium of a heart, a physician typically uses a sheath and dilator to support the transseptal puncturing device. Once the puncture has been completed, the sheath and dilator assembly are pushed over top of the puncturing device, through the puncture in the septum, and into the left atrium. In some cases, a physician may not be able to cross the septum as the transition between sheath and dilator may get stuck or snag at the tissue boundary. This results in the sheath having difficulty to cross the septum and, in some cases, may be unable to cross.

The problem of difficult sheath crossing is more prominent in patients with elastic, aneurysmal, or thickened septa or in patients who have undergone repeat ablations. In order to get the sheath across the septum, additional septum tenting, and mechanical force may be required. This excessive force may cause the sheath to “jump” forward the moment it crosses the tissue, which may result in an accidental perforation of the aortic root, left atrial appendage, left atrial wall, pulmonary vein or artery. Pressure build up during additional tenting, as well as the accidental perforation of left atrium heart structures may lead to a cardiac tamponade which is a life threatening complication, leading to an accumulation of fluid within the pericardial cavity around the heart. This buildup of fluid compresses your heart which in turn reduces the amount of blood able to enter your heart.

Considering these complications, there exists a need to provide a novel puncturing device wherein the puncturing device comprises a means to allow a sheath to safely and easily cross the septum without increasing the risk of accidental perforation of the left atrial wall.

### BRIEF DESCRIPTION OF THE DRAWINGS

In order that the invention may be readily understood, embodiments of the invention are illustrated by way of examples in the accompanying figures, in which:

FIG. 1 is an illustration of the distal portion of a puncturing device with an inflatable feature.

FIG. 2a is an illustration of the puncturing device after being advanced through a successful puncture, prior to expanding the inflatable feature.

FIG. 2b is an illustration of the puncturing device with the inflatable feature expanded such that it dilates the tissue.

FIG. 3 is an illustration of the puncturing device with the inflatable feature expanded in a pulmonary vein, creating an anchor.

FIG. 4a is an illustration of a longitudinal cross section of the puncturing device wherein the inflatable feature is outside the layer of the electrically insulating material.

FIG. 4b is an illustration of a lateral cross section of the puncturing device where the lumen surrounds the electrically insulating layer.

FIG. 4c is an illustration of a lateral cross section of the puncturing device where there is a single lumen exterior of the electrically insulating layer.

FIG. 5a is an illustration of a longitudinal cross section of the puncturing device wherein the inflatable feature is beneath a layer of the electrically insulating material.

FIG. 5b is an illustration of a lateral cross section of the puncturing device where the lumen surrounds the core wire.

FIG. 5c is an illustration of a lateral cross section of the puncturing device where there is a single lumen exterior of the core wire.

FIG. 6 is an illustration of the proximal end of the puncturing device.

### DETAILED DESCRIPTION

After successfully puncturing the septum during a transseptal puncture, a sheath and dilator assembly are progressed through the puncture. The dilator dilates the puncture such that the sheath is able to cross. However, in some cases, a physician may not be able to cross the septum as the transition between sheath and dilator may get stuck or snag at the tissue boundary. This may result in the sheath having difficulty to cross the septum and, in some cases, may be unable to cross. In order to overcome this difficulty, additional tenting and mechanical force may be required. This excessive force may lead to the sheath “jumping” forward once it is able to cross the septum. Sheath jumping may lead to perforations in the left atrial wall which, in combination with the additional mechanical force, may lead to a cardiac tamponade.

The problem of inadvertent perforation of the left atrial wall due to sheath jumping is solved by providing a puncturing device with an inflatable distal feature. The inflatable distal feature may be used to dilate the puncture in the septum such that the sheath is able to cross without snagging on the transition between the sheath and dilator. Alternatively, the inflatable distal feature enables the physician to anchor the puncture device in the pulmonary vein. The puncturing device may then be used as a guiderail, preventing unexpected sheath movement while crossing such that if the sheath does jump forward, it will jump along the guiderail and not inadvertently puncturing the left atrial wall.

In one broad aspect, embodiments of the present invention comprise a device for creating a puncture in a tissue comprising: an elongate member having a puncturing portion at a distal tip; at least one inflatable feature positioned around an outer circumference of the elongate member positioned at a distal portion of the elongate member; and a lumen extending from a proximal end of the elongate member to the inflatable feature, whereby the lumen is in fluid communication with the inflatable feature.

As a feature of this broad aspect, the puncturing portion comprises an energy deliver device capable of delivering energy to the tissue.

As another feature of this broad aspect, the at least one inflatable feature is a balloon. In some embodiments, the balloon is cylindrical. In alternative embodiments, the balloon is spherical. In another embodiment, the balloon is conical. In some embodiments, the balloon is composed of a semi-compliant material. As another feature of this embodiment, the balloon inflates to a diameter of 4 mm. In an alternative embodiment, the balloon is composed of a non-compliant material. As a feature of this embodiment, the balloon inflates to a diameter of 25 mm.

Another feature of this broad aspect, the elongate member of the puncturing device comprises a layer of insulation. In some embodiments, the layer of insulation is positioned overtop the inflatable feature. In an alternative embodiment, the layer of insulation is positioned underneath the inflatable feature.

As a feature of this broad aspect, the elongate member comprises a core wire. In some embodiments, the lumen is positioned such that it surrounds the core wire. In an alternative embodiment, the lumen is a single lumen tube positioned on the outer surface of the core wire.

As another feature of this broad aspect, the device further comprises at least one radiopaque marker. In some embodiments, the at least one radiopaque marker is positioned proximal to the inflatable feature. In another embodiment, the at least one radiopaque marker is positioned distal the inflatable feature. In an alternative embodiment, the device two radiopaque markers, wherein one radiopaque marker is positioned proximal the inflatable feature and the other radiopaque marker is positioned distal the inflatable feature.

In a further broad aspect of the present invention, a method for dilating a puncture in a tissue comprises the steps of: advancing a puncturing device comprising an inflatable feature to a target location on the tissue; puncturing the target location with the puncturing device; advancing the puncturing device through the puncture; positioning the inflatable feature of the puncturing device such that it is in the puncture; and, inflating the inflatable feature such that the puncture dilates.

As a feature of this broad aspect, the method further comprises a step of visualizing and confirming the position of the inflatable feature using at least one radiopaque marker positioned relative to the inflatable feature prior to the step of inflating the inflatable feature.

In a further broad aspect of the present invention, a method for anchoring a puncturing device in a heart structure comprises the steps of: advancing the puncture device comprising an inflatable feature to a target location on the tissue; puncturing the target location with the puncturing device; advancing the puncturing device through the puncture such that it is positioned in the heart structure; and, inflating the inflatable feature such that the inflatable feature acts as an anchor for the puncturing device.

As a feature of this broad aspect, the method further comprises a step of visualizing and confirming the position of the inflatable feature using at least one radiopaque marker positioned relative to the inflatable feature prior to the step of inflating the inflatable feature.

With specific reference now to the drawings in detail, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

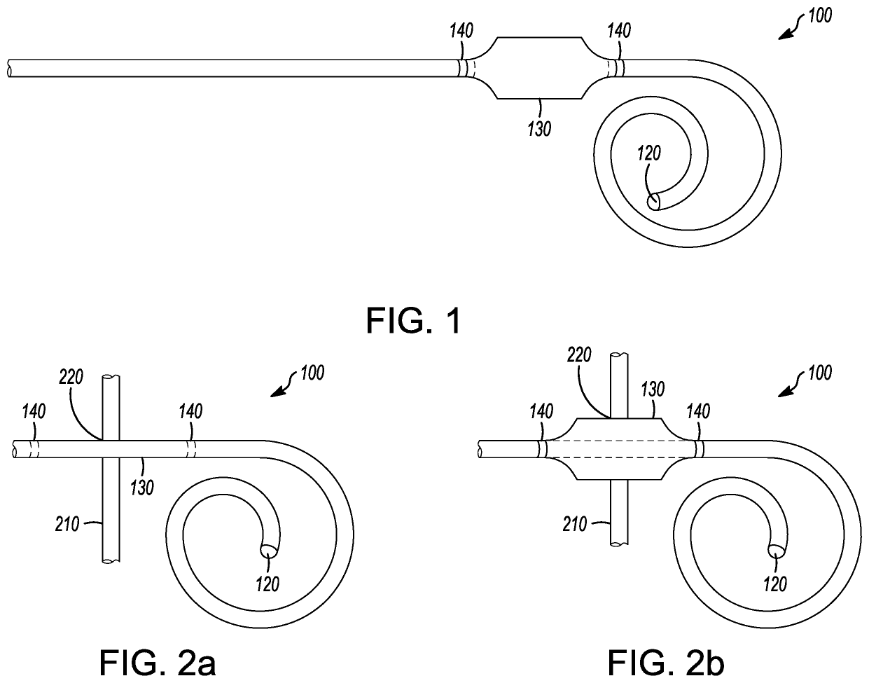

FIG. 1 illustrates an embodiment of an exemplary puncturing device 100 that may be used to access the left atrium via transseptal puncture. The puncturing device 100, for example a pig-tail wire or J-tip wire, is configured to deliver energy to a tissue such as the atrial septum of a patient's heart. The puncturing device 100 comprises an energy delivery device 120, such as an electrode, capable of delivering energy (i.e., radiofrequency energy) to the tissue. In an alternative embodiment, the puncturing device 100 may comprise a mechanical puncturing mechanism, such as a sharp distal tip (not shown). An inflatable feature 130, such as a balloon, is positioned at a distal portion of the puncturing device 100, around the outer circumference of the puncturing device 100. In an alternative embodiment, there may be multiple inflatable features (not shown) positioned at the distal portion.

The inflatable feature 130 may be comprised of a semi-compliant or non-compliant material; the material choice is dependent upon the use of the device. For example, a semi-compliant inflatable feature 130 may be used to anchor the puncturing device 100 into a heart feature, such as a pulmonary vein, such that the puncturing device 100 acts as a guiderail during sheath crossing. The semi-compliant inflatable feature 130 may be composed of Pebax®, engineered nylons (e.g., Grilamid® and Vestamid®), polyethylene terephthalate (PET), or urethane, for example. Alternatively, a non- compliant inflatable feature 130 may be used to dilate the septum after puncture, expanding the hole so that a sheath is able to cross easily and safely. The non-compliant inflatable feature 130 may be comprised of Nylon 12, polyamides, or PET, for example. The shape of the inflatable feature 130 may vary as well. In embodiments where the inflatable feature 130 is used for anchoring the puncturing device 100, the inflatable feature 130 may be spherical or cylindrical in shape. In alternative embodiments where the inflatable feature 130 is used for dilating the puncture, the inflatable feature may be spherical, cylindrical, or conical in shape.

The puncturing device 100 may comprise a means to aid in positioning of the inflatable feature 130. The puncturing device 100 may comprise one or more radiopaque markers 140, located relative to the inflatable feature 130. For example, radiopaque markers 140 may be placed proximal and distal to the inflatable features 130 to provide physician with information on the positioning of the inflatable feature 130 relative to structures in the heart.

As previously described above, the puncturing device 100 may be used to dilate the septum; this will expand the size of the puncture in the septum such that a sheath (not shown) is able to cross easily, without the need of forward force or tenting. Additionally, since the puncturing device 100 comprises a dilation features, there is no need for a stand-alone (separate or self-contained) dilator known in the prior art, which eliminates the transition area between the dilator and sheath which has been found to snag while crossing the septum. In another embodiment, the inflatable feature 130 of the puncturing device 100 may be composed of a non-compliant material to ensure that the inflatable feature 130 remains rigid, resisting the force of the septum 210 onto the inflatable feature 130, while expanding, and dilating, the puncture outward. The inflatable feature 130 inflates to a sufficient size, such as to a diameter of between the range of 4-10 mm, to facilitate dilation of the septum tissue to be punctured. With reference now to FIG. 2a, the puncturing device 100 delivers energy to the tissue of the septum 210 which creates a hole or passage 220. During this time, the inflatable feature 130 is in an uninflated state. The puncturing device 100 is pushed through the hole 220 and positioned such that the inflatable feature 130 (in an uninflated state) is in the hole 220. In one example, positioning of the device may be achieved through the use of radiopaque markers 140, these markers 140 may be positioned such that they are located proximal and distal to the inflatable feature 130; using various imaging techniques, such as fluoroscopy, the physician is able to visualize the markers 140. When one radiopaque marker is in the right atrium while the other is in the left atrium, the inflatable feature 130 is in the correct position. The inflatable feature 130 is then inflated, expanding, once it is in the correct position. This expansion, will dilate the hole 220 in the septum 210, as depicted in FIG. 2b.

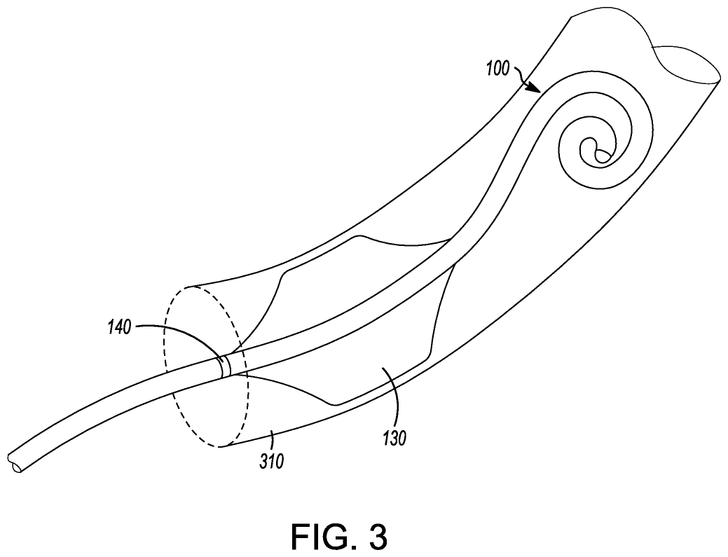

The puncturing device 100 may be used as a guiderail for advancement of the sheath into the left atrium by anchoring into a heart feature. For example, upon a successful puncture, the puncturing device 100 may be anchored into a pulmonary vein in the left atrium. In another embodiment, the inflatable feature 130 of the puncturing device 100 may be composed of a semi-compliant material as to not damage the heart structure. The inflatable feature 130 may inflate to a sufficient size, preferably within the range of 20-30 mm in diameter, for example, a diameter of 25 mm, to facilitate anchoring in the heart structure. Upon completing a successful puncture into the septum, the puncturing device 100 is pushed through into the left atrium with the inflatable feature 130 in an uninflated state. The puncturing device 100 is then advanced into a pulmonary vein 310 where the inflatable feature 130 is inflated, creating an anchoring point as illustrated in FIG. 3. The sheath is advanced along the puncturing device 100 through the puncture. Anchoring the puncturing device 100 and using it as a guiderail will prevent unexpected jumping or movement as the sheath is advanced into the left atrium. When positioning the puncturing device 100 into the pulmonary vein 310, one or more radiopaque markers 140, in conjunction with various visualization methods (i.e., fluoroscopy), may be used to indicate the location of the inflatable feature 130 on the puncturing device 100. For example, a radiopaque marker 140 may be positioned proximal the inflatable feature 130; once the radiopaque marker 140 is in the pulmonary vein 310, the physician will know that the inflatable feature 130 is located in the pulmonary vein 310. In alternative embodiments, a radiopaque marker 140 may be positioned distal the inflatable feature 130. Alternatively, a radiopaque marker 140 may be positioned distal the inflatable feature 130 while another radiopaque marker 140 may be positioned proximal the inflatable feature 130.

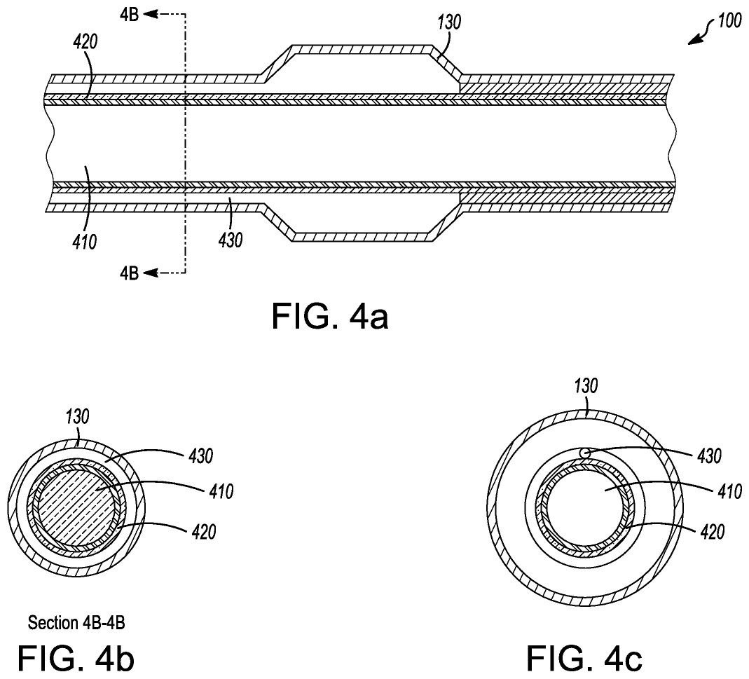

A longitudinal cross-section of the puncturing device 100 is illustrated in FIG. 4a. The puncturing device 100 may be a pigtail or J-tip wire configured for delivering energy to the tissue. The puncturing device 100 should be flexible to provide the ability to navigate difficult anatomy while advancing through a body lumen and may be manipulated through difficult or tortuous anatomy. The puncturing device 100 should also be rigid enough to allow for advancement of the sheath overtop of the puncturing device 100. In some embodiments, the body of the puncturing device 100 may be comprised of a core wire 410. The core wire 410 may be comprised of an electrically conductive material such that energy can be delivered from an energy generator, along the length of the puncturing device, and delivered to the tissue from the energy delivery device at the distal tip, such materials may include nitinol or stainless steel. The core wire 410 may have an electrically insulative layer 420, wherein the energy delivery device may be created by leaving a portion of the distal tip exposed. The electrically insulative layer 420 may be applied as a spray coating or a heat shrink wrap. Materials for the electrically insulative layer 420 may be polyetheretherketone (PEEK) or polyimide, for example. In some embodiments, such as those illustrated in FIG. 4a-4c, the inflatable feature 130 may be placed over top of the electrically insulative layer 420. The inflatable feature 130 may be expanded by injecting saline, for example, through a lumen 430 that is in fluid communication with the inflatable feature 130. The lumen 430 may be positioned such that it surrounds the circumference of the core wire 410, as illustrated in FIG. 4b. In an alternative embodiment, the lumen 430 may be positioned as a single lumen on the outside of the electrically insulating material 420; this embodiment is illustrated in FIG. 4c.

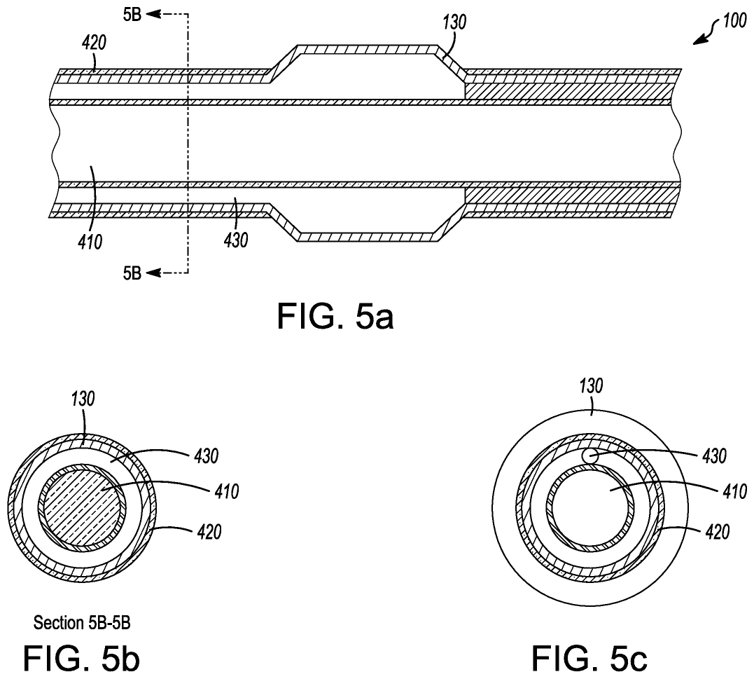

An alternative embodiment of the invention can be seen in FIG. 5a-5c. The inflatable feature 430 may be placed underneath the electrically insulative layer 420. FIG. 5a illustrates a longitudinal cross section of the puncturing device 100. The inflatable feature 130 is located beneath the electrically insulative layer 420 such that the inflation or expansion of the inflatable feature 130 is not obstructed by the electrically insulative layer 420. For example, the insulative layer 420 may be applied such that it leaves a gap in which the inflatable feature 130 may be positioned. Alternatively, in some embodiments, the insulative layer 420 may be cut such that the inflatable feature 130 is able to expand outwards. With reference now to FIG. 5b, in some embodiments the lumen 430, which is in fluid communication with the inflatable feature 130, may be positioned such that it surrounds the circumference of the core wire 410. Alternatively, the lumen 430 may be positioned as a single lumen on the outside of the core wire 410, as seen in FIG. 5c.

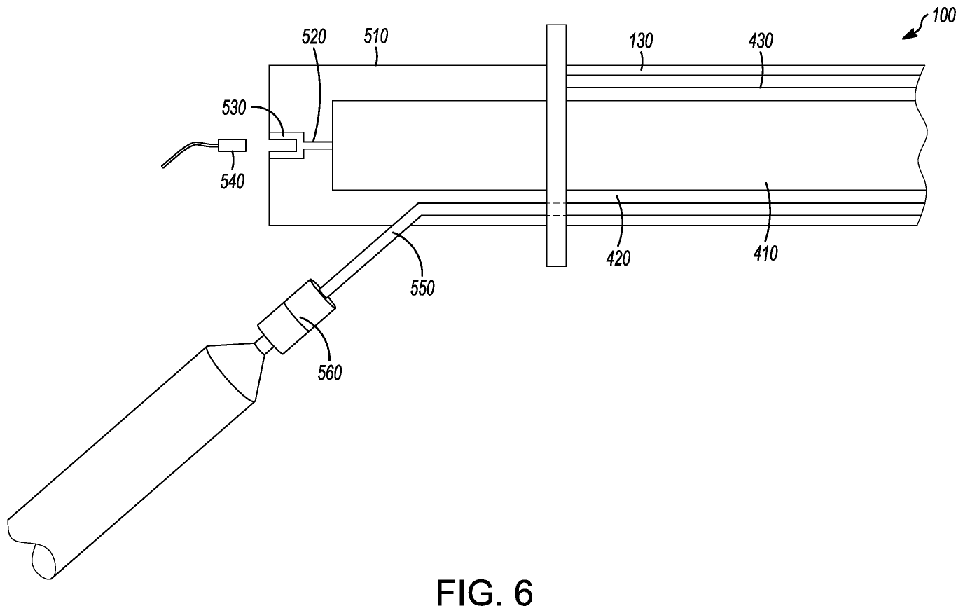

An example of the proximal end of the puncturing device 100 is illustrated in FIG. 6. In some embodiments, the proximal end of the puncturing device 100 may be coupled to a hub 510, which may be structured to electrically couple the core wire 410 to an energy source, such as a generator. In one embodiment the hub 510 may comprise a conductive wire 520 connected to the end of the core wire 410, for example by welding or brazing. The other end of the conductive wire 520 may be coupled to a connector 530, such as a banana jack, which a banana plug 540 may be inserted to electrically couple to an energy source. This connection allows energy to be delivered from the energy source, through the plug 540, jack 530, and conductive wire 520 to the core wire 410 and to the energy delivery device 120 and, finally, to the tissue. The hub 510 may further comprise tubing 550 which is in fluid communication with the lumen 430 of the inflatable feature. The tubing may be structured to be operatively coupled 560 at one of thereof to a source of fluid 570, such as a syringe. This coupling may be in the form of a Tuohy-Borst adapter.

The embodiments of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

We claim:

1. A device for creating a puncture in a tissue comprising:
an elongate member having a puncturing portion at a distal tip;
at least one inflatable feature positioned around an outer circumference of the elongate member positioned at a distal portion of the elongate member; and
a lumen extending from a proximal end of the elongate member to the inflatable feature, wherein the lumen is in fluid communication with the inflatable feature.

2. The device of claim 1, wherein the puncturing portion comprises an energy delivery device capable of delivering energy to the tissue.

3. The device of claim 1, wherein the at least one inflatable feature is a balloon.

4. The device of claim 3, wherein the balloon is composed of a semi-compliant material.

5. The device of claim 4, wherein the balloon inflates to a diameter of 4 mm.

6. The device of claim 3, wherein the balloon is composed of a non-compliant material.

7. The device of claim 6, wherein the balloon inflates to a diameter of 25 mm.

8. The device of claim 1, wherein the elongate member comprises a layer of insulation.

9. The device of claim 8, wherein the layer of insulation is positioned overtop the inflatable feature.

10. The device of claim 8, wherein the layer of insulation is positioned underneath the inflatable feature.

11. The device of claim 1, wherein the elongate member comprises a core wire and wherein the lumen is positioned such that it surrounds the core wire.

12. The device of claim 1, wherein the elongate member comprises a core wire and wherein the lumen is a single lumen tube positioned on the outer surface of the core wire.

13. The device of claim 1, wherein the device further comprises at least one radiopaque marker.

14. The device of claim 13, wherein the at least one radiopaque marker is positioned proximal to the inflatable feature.

15. The device of claim 13, wherein the at least one radiopaque marker is positioned distal to the inflatable feature.

16. The device of claim 13, wherein the device comprises two radiopaque markers, wherein one radiopaque marker is positioned proximal the inflatable feature and the other radiopaque marker is positioned distal the inflatable feature.

17. A method for dilating a puncture in a tissue, comprising:
advancing a puncturing device comprising an inflatable feature to a target location on the tissue;
puncturing the target location with the puncturing device to define a puncture;
advancing the puncturing device through the puncture;
positioning the inflatable feature of the puncturing device such that it is in the puncture; and,
inflating the inflatable feature such that the puncture dilates.

18. The method of claim 17, further comprising visualizing and confirming the position of the inflatable feature using at least one radiopaque marker positioned relative to the inflatable feature prior to inflating the inflatable feature.

19. A method for anchoring a puncturing device proximate a heart structure, the method comprising:
advancing the puncture device comprising an inflatable feature to a target location on the tissue;
puncturing the target location with the puncturing device to define a puncture;
advancing the puncturing device through the puncture such that it is positioned in the heart structure; and,
inflating the inflatable feature such that the inflatable feature acts as an anchor for the puncturing device.

20. The method of claim 19, wherein the method further comprises visualizing and confirming the position of the inflatable feature using at least one radiopaque marker positioned relative to the inflatable feature prior inflating the inflatable feature.
